(19) United States
(12) Patent Application Publication
(10) Pub. No.: US 2016/0367404 A1
(43) Pub. Date: Dec. 22, 2016

(54) Otic Extraction Assembly

(71) Applicant: Richard Shomo, Fair Oaks Ranch, TX (US)

(72) Inventor: Richard Shomo, Fair Oaks Ranch, TX (US)

(21) Appl. No.: 14/744,882

(22) Filed: Jun. 19, 2015

(51) Int. Cl. A61F 11/00; A61B 1/227; A61M 1/00

(52) U.S. Cl. CPC A61F 11/006; A61M 1/0039; A61B 1/227

(57) ABSTRACT

An otic extraction assembly includes a speculum that has a horn portion and a tube portion. The speculum is substantially hollow and the tube portion may be inserted into an ear canal of a user. The horn portion may insertably receive an otoscope thereby facilitating the user's ear canal to be visually inspected. A suction tube is positioned within the tube portion. The suction tube may be fluidly coupled to a suction source thereby facilitating foreign objects to be suctionally removed from the user's ear canal.

### BACKGROUND OF THE DISCLOSURE

### Field of the Disclosure

The disclosure relates to speculum devices and more particularly pertains to a new speculum device for suctionally removing foreign objects from an ear canal of a user.

### SUMMARY OF THE DISCLOSURE

An embodiment of the disclosure meets the needs presented above by generally comprising a speculum that has a horn portion and a tube portion. The speculum is substantially hollow and the tube portion may be inserted into an ear canal of a user. The horn portion may insertably receive an otoscope thereby facilitating the user's ear canal to be visually inspected. A suction tube is positioned within the tube portion. The suction tube may be fluidly coupled to a suction source thereby facilitating foreign objects to be suctionally removed from the user's ear canal.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a perspective view of an otic extraction assembly according to an embodiment of the disclosure.

FIG. 2 is a front view of an embodiment of the disclosure.

FIG. 3 is a cross sectional view taken along line 3-3 of FIG. 1 of an embodiment of the disclosure.

FIG. 4 is a right side view of an embodiment of the disclosure.

FIG. 5 is a perspective in-use view of an embodiment of the disclosure.

### DESCRIPTION OF THE PREFERRED EMBODIMENT

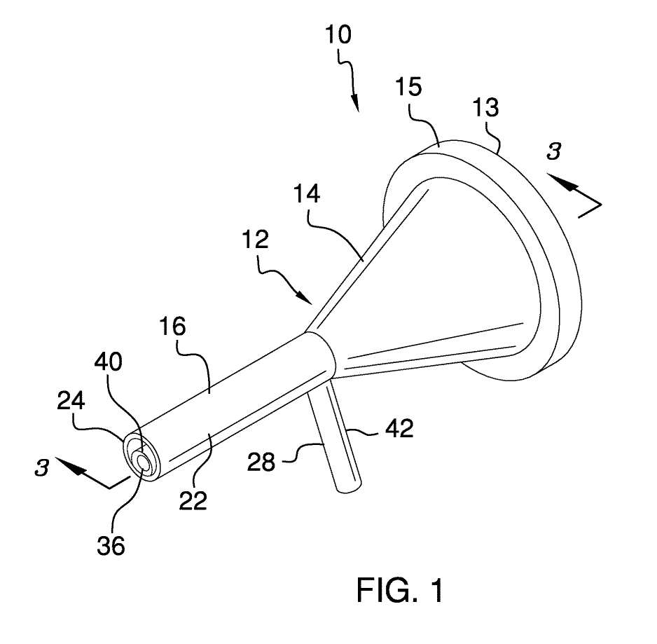

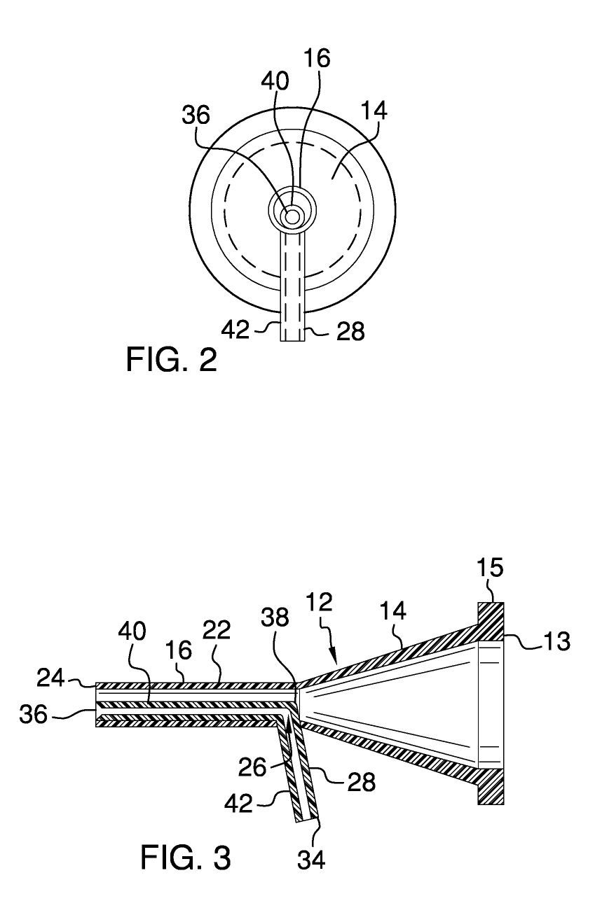

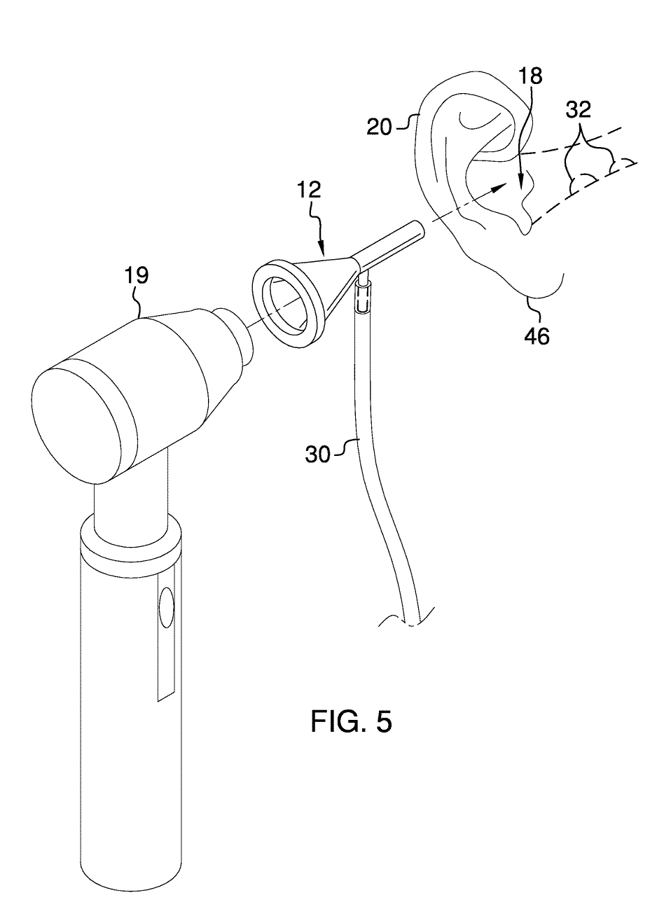

With reference now to the drawings, and in particular to FIGS. 1 through 5 thereof, a new speculum device embodying the principles and concepts of an embodiment of the disclosure and generally designated by the reference numeral 10 will be described.

As best illustrated in FIGS. 1 through 5, the otic extraction assembly 10 generally comprises a speculum 12 that has a horn portion 14 and a tube portion 16. The speculum 12 is substantially hollow and the tube portion 16 may be inserted into an ear canal 18 of a user 20. The horn portion 14 may insertably receive an otoscope 19 thereby facilitating the user's ear canal 18 to be visually inspected. The horn portion 14 has a first end 13 and the first end 13 is open. A lip 15 is coupled to the horn portion 14 and the lip 15 extends outwardly from the first end 13.

The tube portion 16 has an outer wall 22 and the tube portion 16 has a distal end 24 with respect to the horn portion 14. The distal end 24 is open and the outer wall 22 has an opening 26 extending therethrough. The opening 26 is positioned adjacent to an intersection of the tube portion 16 and the horn portion 14. The tube portion 16 may be comprised of a resiliently flexible material such as rubber or the like. The tube portion 16 may have an outside diameter ranging between approximately 2 mm and 4 mm thereby facilitating the tube portion 16 to fit within a variety of sizes of ear canals 18.

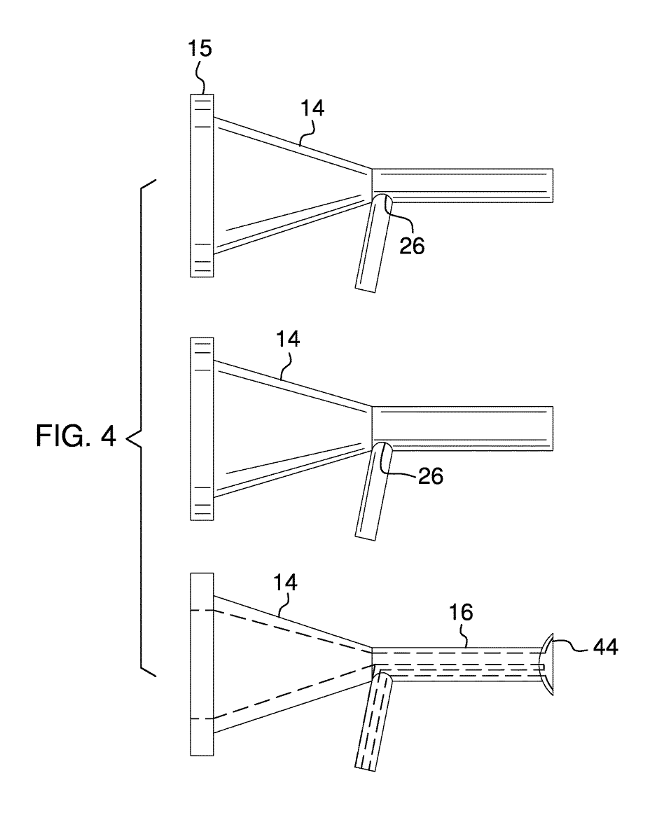

A suction tube 28 is provided and the suction tube 28 is positioned within the tube portion 16. The suction tube 28 may be fluidly coupled to a suction source 30 thereby facilitating foreign objects 32 to be suctionally removed from the user's ear canal 18. The foreign objects 32 may be collections of ear wax or the like. The suction tube 28 has a first end 34, a second end 36 and a bend 38 positioned thereon. The bend 38 is positioned between the first end 34 and the second end 36 to define a first portion 40 of the suction tube 28 forming an angle with respect to a second portion 42 of the suction tube 28. The angle may be an angle ranging between approximately 100° and 90°. As shown in FIG. 4, a cup 44 is provided that may be coupled to the distal end 24 of the tube portion 16. The cup 44 is open such that the cup 44 is in fluid communication with the suction tube 28. The cup 44 may abut the user's ear 46 thereby facilitating an air tight seal between the cup 44 and the user's ear 46.

The suction tube 28 is positioned within the opening 26 having the first portion 40 of the suction tube 28 being positioned within the tube portion 16. Thus, the second end 36 is aligned with the distal end 24. The second portion 42 of the suction tube 28 extends downwardly from the tube portion 16. The second end 36 may be fluidly coupled to the suction source 30 such that the first end 34 suctionally removes the foreign objects 32 from the user's ear canal 18.

In use, the suction source 30 is fluidly coupled to the second end 34 of the suction tube 28. The distal end 24 of the tube portion 16 is inserted into the user's ear canal 18. The otoscope 19 is inserted into the first end 13 of the horn portion 14 and the user's ear canal 18 is visually inspected. The second end 36 of the suction tube 28 suctionally removes the foreign objects 32 from the user's ear canal 18.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An otic extraction assembly configured to remove foreign objects from an ear canal of a user, said assembly comprising:
a speculum having a horn portion and a tube portion, said speculum being substantially hollow, said tube portion being configured to be inserted into an ear canal of a user, said horn portion being configured to insertably receive an otoscope thereby facilitating the user's ear canal to be visually inspected; and
a suction tube being positioned within said tube portion, said suction tube being configured to be fluidly coupled to a suction source thereby facilitating foreign objects to be suctionally removed from the user's ear canal.

2. The assembly according to claim 1, wherein tube portion has an outer wall, said tube portion having a distal end with respect to said horn portion, said distal end being open, said outer wall having an opening extending therethrough, said opening being positioned adjacent to an intersection of said tube portion and said horn portion.

3. The assembly according to claim 1, wherein said suction tube has a first end and a second end, said suction tube having a bend thereon, said bend being positioned between said first end and said second end to define a first portion of said suction tube forming an angle with respect to a second portion of said suction tube.

4. The assembly according to claim 3, wherein:
said tube portion has an opening and a distal end; and
said suction tube being positioned within said opening having said first portion of said suction tube being positioned within said tube portion such that said second end is aligned with said distal end.

5. The assembly according to claim 4, wherein said second portion of said suction tube extends downwardly from said tube portion, said second end being configured to be fluidly coupled to the suction source such that said first end suctionally removes the foreign objects from the user's ear canal.

6. An otic extraction assembly configured to remove foreign objects from an ear canal of a user, said assembly comprising:
a speculum having a horn portion and a tube portion, said speculum being substantially hollow, said tube portion being configured to be inserted into an ear canal of a user, said horn portion being configured to insertably receive an otoscope thereby facilitating the user's ear canal to be visually inspected, said tube portion having an outer wall, said tube portion having a distal end with respect to said horn portion, said distal end being open, said outer wall having an opening extending therethrough, said opening being positioned adjacent to an intersection of said tube portion and said horn portion; and
a suction tube being positioned within said tube portion, said suction tube being configured to be fluidly coupled to a suction source thereby facilitating foreign objects to be suctionally removed from the user's ear canal, said suction tube having a first end and a second end, said suction tube having a bend thereon, said bend being positioned between said first end and said second end to define a first portion of said suction tube forming an angle with respect to a second portion of said suction tube, said suction tube being positioned within said opening having said first portion of said suction tube being positioned within said tube portion such that said second end is aligned with said distal end, said second portion of said suction tube extending downwardly from said tube portion, said second end being configured to be fluidly coupled to the suction source such that said first end suctionally removes the foreign objects from the user's ear canal.
